(19) United States
(12) Patent Application Publication
(10) Pub. No.: US 2026/0207346 A1
(43) Pub. Date: Jul. 23, 2026

(54) ARTIFICIAL VERTEBRAE, ARTIFICIAL SPINES MADE THEREOF, AND RELATED METHODS

(71) Applicant: HIS MAJESTY THE KING IN RIGHT OF CANADA, AS REPRESENTED BY THE MINISTER OF NATIONAL DEFENCE, Ottawa (CA)

(72) Inventors: Robert Durocher, Québec (CA); Georges Menard, Québec (CA)

(21) Appl. No.: 19/137,902

(22) PCT Filed: Dec. 6, 2023
(86) PCT No.: PCT/IB2023/062324

§ 371 Date: Jun. 11, 2025

(30) Foreign Application Priority Data
Dec. 12, 2022 (CA) 3185266

(51) Int. Cl. A61F 2/44; A61F 2/30; A61F 2/46

(52) U.S. Cl. CPC A61F 2/4425; A61F 2/4657; A61F 2002/30281; A61F 2002/443; A61F 2002/449; A61F 2002/4666

(57) ABSTRACT

An artificial vertebra includes a body of rigid material. The body includes a disc-contact surface shaped to contact an artificial intervertebral disc and an instrumentation surface shaped to receive a sensor. A biofidelic instrumented spine, such as an artificial lumbar spine, includes a series of such rigid artificial vertebrae arranged in a column with resilient artificial intervertebral discs positioned between the rigid artificial vertebrae. Artificial ligaments may be attached to the artificial vertebrae. A sensor, such as a strain gauge or pressure mapping sensor, may be positioned at a rigid artificial vertebra or at multiple rigid artificial vertebrae to measure an internal loading.

### CROSS-REFERENCE TO RELATED APPLICATIONS

This application claims priority to Canadian patent application 3,185,266, filed Dec. 12, 2022, the entirety of which is incorporated herein by reference.

### TECHNICAL FIELD

The present disclosure relates to an artificial spine and artificial vertebrae.

### BACKGROUND

People may suffer spinal injury from a large or sudden force. Members of the armed forces may be exposed to such loading, such as during an underbelly blast (UBB) event, during use of an aircraft ejection seat, or while riding in small marine vessels. Vehicle occupants may be subjected to acceleration or impact injuries that affect the spine. Often such injuries are located at the lumbar spine and may include squeletic injuries and soft-tissue injuries.

One known methodology for assessing injury and the performance of protective equipment for vehicle occupants subjected to a UBB threat is described in NATO AEP-55 Vol. 2. This methodology is widely used among NATO countries as a test standard for qualifying new equipment. It specifies using a Dynamic Response Index (DRI) model to assess risk of pelvis and lumbar region injury. The DRI model was proposed by Stech & Payne in 1969. The DRI model is based on a simple lump mass model of the spine and was developed to assess the risk of spinal compression injuries sustained during aircraft ejection events. The original model uses a seat pan vertical acceleration as the input excitation. AEP-55 Vol. 2 uses a Hybrid III Anthropomorphic Test Device (ATD) pelvis acceleration in the z-direction as the input to compute the DRI model. A computed DRI value is compared to a threshold value to determine a probability of injury.

DRI may fail to accurately predict spinal injuries, such as those caused by UBB events, for example, when complex lumbar loading is expected and when the person's sitting posture is different from the 90 -90 posture (i.e., lower legs and back at 90 degrees to upper leg) that was used to develop this model. In addition, lumbar load criteria that is based on a maximum acceptable compression force and often measured using a FAA Hybrid III anthropomorphic test device (ATD or crash test dummy) is typically limited to assessing load in the vertical direction and does not take into consideration bending or shear loads that are present in the lumbar spine in most UBB events. A single-axis model lacks fidelity.

A known multi-axis model is the multiaxial dynamic response criteria, also referred as the Brinkley Dynamic Response Criteria (BDRC) developed by NASA. This model is similar to the DRI but uses three lump masses models, one for each of the three orthogonal spine axes. However, this model still requires a simplification of human and vehicle interactions to solely an acceleration input model, which is insufficient and may neglect important injury mechanisms, such as bending loading that can be observed in the lumbar spine. In addition, this model does not consider spine geometry and loading distribution throughout the spine vertebrae and their different anatomical regions, thus limiting its capability to assess injury types and mechanisms.

### SUMMARY

The present disclosure provides techniques to solve the above discussed problems with injury assessment models and methods, so as to reduce the incidence of lumbar spine injuries. The present disclosure provides a more biofidelic representation of the lumbar spine that more closely represents human lumbar spine geometry and reaction under load for various postures.

According to an aspect of the present disclosure, an artificial vertebra includes a body of rigid material. The body includes a disc-contact surface shaped to contact an artificial intervertebral disc and an instrumentation surface shaped to receive a sensor.

The body may further include a main part and an end plate that defines the disc-contact surface. The end plate may be removably attachable to the main part.

The end plate may be a first end plate that is removably attachable to a first side of the main part. The body may further include a second end plate that is removably attachable to a second side of the main part that is opposite the first side.

The main part and the end plate may be shaped to provide a void between the main part and the end plate when attached. The artificial vertebra may further include artificial cancellous bone shaped to be captured within the void between the end plate and the main part.

The end plate may be tapered in a planar direction to provide lordosis to an assembly of artificial vertebrae that includes the artificial vertebra.

The body may further include a pair of opposing planar bodies connected by a post. The post may include the instrumentation surface.

The body may further include an artificial pedicle. The artificial pedicle may include the instrumentation surface.

The body may further include an attachment point for an artificial ligament.

The artificial vertebra may further include a posterior component extending from the body and shaped to simulate additional posterior structure of a vertebra. The posterior component may include an attachment point for an artificial ligament.

According to another aspect of the present disclosure, a biofidelic instrumented spine includes a plurality of rigid artificial vertebrae arranged in a column and a plurality of resilient artificial intervertebral discs. Each of the plurality of resilient artificial intervertebral discs is positioned between adjacent vertebrae of the plurality of rigid artificial vertebrae. The biofidelic instrumented spine further includes a sensor positioned at a rigid artificial vertebra of the plurality of rigid artificial vertebrae to measure an internal loading of the rigid artificial vertebra.

The biofidelic instrumented spine may further include an artificial ligament connected to each of the plurality of rigid artificial vertebrae.

Each of the plurality of rigid artificial vertebrae may be made of metal.

Each resilient artificial intervertebral disc may be made of elastomer.

The sensor may include a strain gauge.

The biofidelic instrumented spine may further include artificial cancellous bone within the rigid artificial vertebra.

The sensor may include a pressure mapping sensor.

The biofidelic instrumented spine may include a plurality of sensors each positioned at a respective rigid artificial vertebra of the plurality of rigid artificial vertebrae.

Each of the plurality of rigid artificial vertebrae may include a tapered part to provide lordosis to the biofidelic instrumented spine.

According to another aspect of the present disclosure, a method includes applying an external load to a biofidelic instrumented spine that includes a plurality of rigid artificial vertebrae arranged in a column with resilient artificial intervertebral discs positioned therebetween. The rigid artificial vertebrae are connected by an artificial ligament. The method further includes measuring an internal load using a sensor positioned at a rigid artificial vertebra of the plurality of rigid artificial vertebrae.

The external load may be configured to simulate an underbelly blast event.

### BRIEF DESCRIPTION OF THE DRAWINGS

FIG. 1 is a perspective view of an example biofidelic instrumented spine according to the present disclosure.

FIG. 2 is a front view of the example biofidelic instrumented spine of FIG. 1.

FIG. 3 is a side view of the example biofidelic instrumented spine of FIG. 1.

FIG. 4 is an exploded perspective view of an example artificial vertebra according to the present disclosure.

FIG. 5 is a perspective view of the example artificial vertebra of FIG. 4 assembled.

FIG. 6 is an exploded perspective view of example end plates of FIG. 4 and an example artificial intervertebral disc.

FIG. 7 is a perspective view of the end plates and artificial intervertebral disc of FIG. 6 assembled.

FIG. 8 is a side view of an example end plate according to the present disclosure.

FIG. 9 is a side view of the example biofidelic instrumented spine of FIG. 1 with example angles.

FIG. 10 is an exploded perspective view of an example artificial vertebra with a pressure mapping sensor and strain gauges.

FIG. 11 is a perspective view of the example lower securing member of FIG. 1.

FIG. 12 is a side view of the example biofidelic instrumented spine of FIG. 1 integrated into an anthropomorphic test device.

FIG. 13 is a flowchart of an example method of measuring a vertebral load according to the present disclosure.

### DETAILED DESCRIPTION

The present disclosure relates generally to artificial rigid vertebrae and a biofidelic instrumented spine including such vertebrae. The techniques discussed herein support prediction and the prevention of spinal injuries, particularly lumbar spine injuries. Such injuries may constitute high incidence injury types for vehicle occupants that are exposed to various impacts including underbody blast events, aerospace ejection, and other injuries.

As discussed above, current methods assess risk of injury models and injury risk curves developed for aviation and use measurement devices developed for the automotive industry. These methods have drawbacks as they do not adequately capture the various injury mechanisms that are observed operationally. Also, current methods do not provide a biofidelic representation of the spine geometry in various seating postures. Consequently, current methods show deficiencies for measuring the performance of protection systems to prevent against spine injuries. Other commercial artificial biofidelic spines are available for orthopedic and medical education purposes. These, while realistic in appearance, are not adequate for conducting repetitive comparative tests and do not permit easy measurement of load distributions between the spine components.

The techniques discussed herein may be integrated in existing ATDs or as a standalone Functional Spine Unit (FSU) to conduct research associated with the measurement of spine load distributions. The system allows replicating biofidelic geometries of the spine, particularly the lumbar spine, in various standing and sitting postures. An artificial lumbar spine may include vertebrae L1 to L5 as well as connectors to integrate the lumbar spine with the pelvis and thoracic spine of an ATD. Each vertebra includes, endplates, instrumented vertebrae body allowing measurements of tensile/compressive force, shear force, and bending moment. Pressure sensor film located under the endplate allows measurement of disc pressure distribution on a vertebra body. Vertebra pedicles may be instrumented to measure posterior structure force and moment. Bending moment of the traverse process can be measured. An artificial vertebra posterior structure may include the lamina, spinous process, and upper and lower articular processes.

Artificial vertebrae may be interconnected using elastomer intervertebral discs, artificial longitudinal (anterior and posterior) ligaments, artificial interspinous ligaments, artificial intertransverse ligaments, and artificial articular ligaments. A modular design allows improvement of its components to focus research on specific elements of the spine, as well as simplifying repair and replacement of parts. The instrumentation of the vertebrae allows measuring complex loading distribution in the lumbar spine for various seating postures. In addition, an injury assessment model, using the instrumented vertebrae measurement, may be used to predict injury mechanisms as well as injury severity. The instrumentation may be easily configured or modified to accommodate particular research or test requirements.

The techniques discussed herein provide for biofidelity, both from a geometric and material behavior standpoint. The assembly and range of motion can replicate spine morphology for various sitting postures, allowing measurement of representative and biofidelic loading distribution. As opposed to other models in which only reaction loads at the cranial or caudal are measured, the techniques discussed herein provide measurement of multi-axis loads at the vertebrae level, allowing the identification of the most susceptible area of sustaining injury as well as information for predicting injury mechanisms.

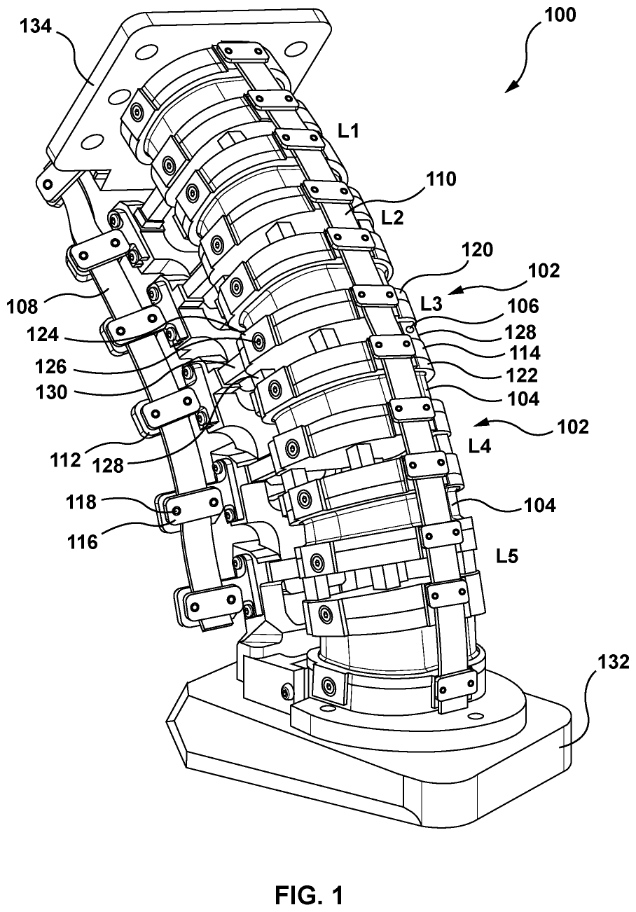

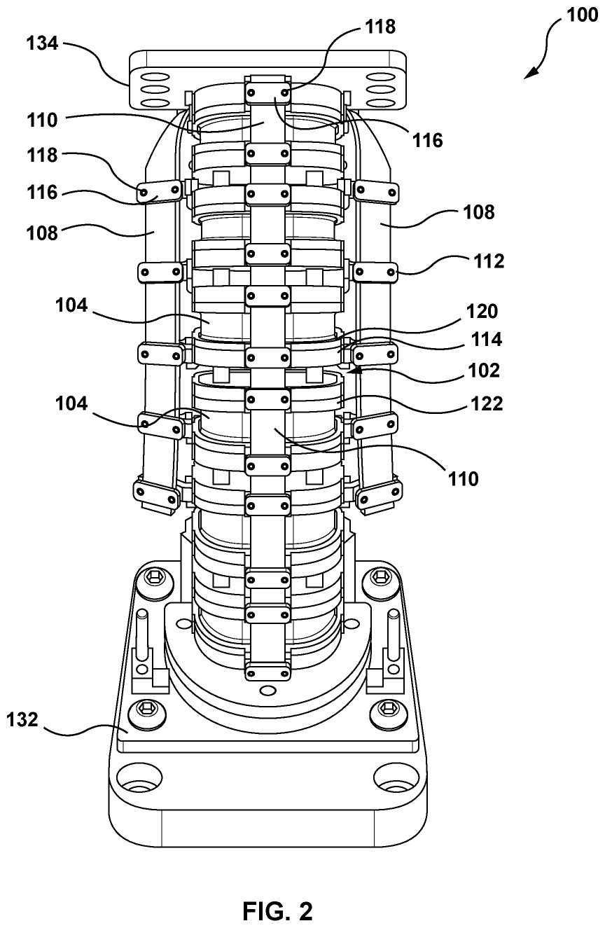

FIGS. 1-3 show an example biofidelic instrumented spine 100. In this example, the biofidelic instrumented spine 100 simulates a human lumbar spine from vertebrae L1 to L5. The techniques discussed herein may be used for any region of a human or animal spine or region thereof.

The biofidelic instrumented spine 100 includes a plurality of rigid artificial vertebrae 102 arranged in a column, a plurality of resilient artificial intervertebral discs (IVD) 104 positioned between the rigid artificial vertebrae 102, and a sensor 106 positioned at a rigid artificial vertebra 102. Any suitable type and number of sensors 106 may be positioned at any of the rigid artificial vertebrae 102.

Each artificial vertebra 102 may have an overall shape that is comparable to a human vertebra and may be primarily or completely made of metal to provide rigidity. Each artificial vertebra 102 may have the same or similar construction of assembled multiple components, so as to simplify repair and replacement.

Each of the resilient artificial intervertebral discs 104 is positioned between an adjacent pair of rigid artificial vertebrae 102. The resilient artificial intervertebral discs 104 may be made of elastomer.

Suitable sensors 106 include a strain gauge (depicted) and a pressure mapping sensor, such as a pressure mapping film. A strain gauge may be affixed to a surface of a rigid artificial vertebra 102. A pressure mapping sensor may be provided integral to a rigid artificial vertebra 102, as will be discussed below.

In this example, the biofidelic instrumented spine 100 further includes artificial intertransverse ligaments (ITL) 108, an artificial anterior longitudinal ligament (ALL) 110, an artificial posterior longitudinal ligament (PLL) 300, and an artificial interspinous and supraspinous ligament (ISL and SSL) 302.

To facilitate biofidelity, the artificial intertransverse ligaments (ITL) 108 extend along opposing posterior sides of the spine 100 attaching to a respective transverse process 112 of each vertebra 102, the artificial anterior longitudinal ligament 110 extends along the front of the spine 100 attaching to the anterior of a main part 114 of each vertebra 102, the artificial posterior longitudinal ligament 300 extends along the back of the spine 100 attaching to the posterior of the main part 114 of each vertebra 102, and the artificial interspinous and supraspinous ligament 302 extends along the back of the spine 100 and attached to an artificial spinous process 304 of each vertebra 102.

In this example, the functions of the ligamentum flavum (LF) and facet capsular ligaments (FCL) are simulated by the artificial interspinous and supraspinous ligament (ISL and SSL). The artificial ligaments 108, 110, 300, 302 may be made of moulded polymeric bands and may be attached to the artificial vertebrae 102 using mechanical compression clamps 116 secured by screws 118.

In this example, each artificial vertebra 102 includes a main part 114 that has the same shape for all artificial vertebrae 102. Further, each artificial vertebra 102 includes opposing end plates 120, 122 that are removably attachable to opposite sides of the main part 114 by, for example, screws. End plates 120, 122 for different artificial vertebrae 102 may have different shapes, so as to provide lordosis (curvature) to the assembled spine 100. Attachment features, such as tabs 124 and screw holes 126, may be provided to the end plates 120, 122 to attach the end plates 120, 122 to the main part 114. Such attachment features may have consistent geometry among end plates 120, 122, whether differently shaped or not, so that any end plate 120, 122 may be attached to any main part 114.

The main part 114 may include a post 128 shaped as a rectangular prism. Any suitable number of posts 128 may be used. In this example four posts 128 arranged in a rectangular pattern are used. A post 128 may provide an instrumentation surface for mounting a sensor 106, such as a strain gauge.

The main part 114 may include an artificial pedicle 130 extending therefrom. The artificial pedicle 130 may be shaped as a rectangular prism. Each artificial vertebra 102 may include two such artificial pedicles 130, as exists in a human vertebra. As with the post 128, an artificial pedicle 130 may include an instrumentation surface for mounting a sensor 106.

The biofidelic instrumented spine 100 may be secured between a lower securing member 132 and an upper securing member 134. A securing member 132, 134 may provide connection points for artificial vertebra 102 and anchor points for artificial ligaments 108, 110, 300, 302. External loads may be applied to the securing member 132, 134 to simulate the effects of a UBB event or similar injurious loading. For example, the lower securing member 132 may be attached to a rigid base, and a load may be applied to the upper securing member 134. When the biofidelic instrumented spine 100 is integrated into an anthropomorphic test device, the securing member 132, 134 may be other components of the ATD or may be attached to other components of the ATD.

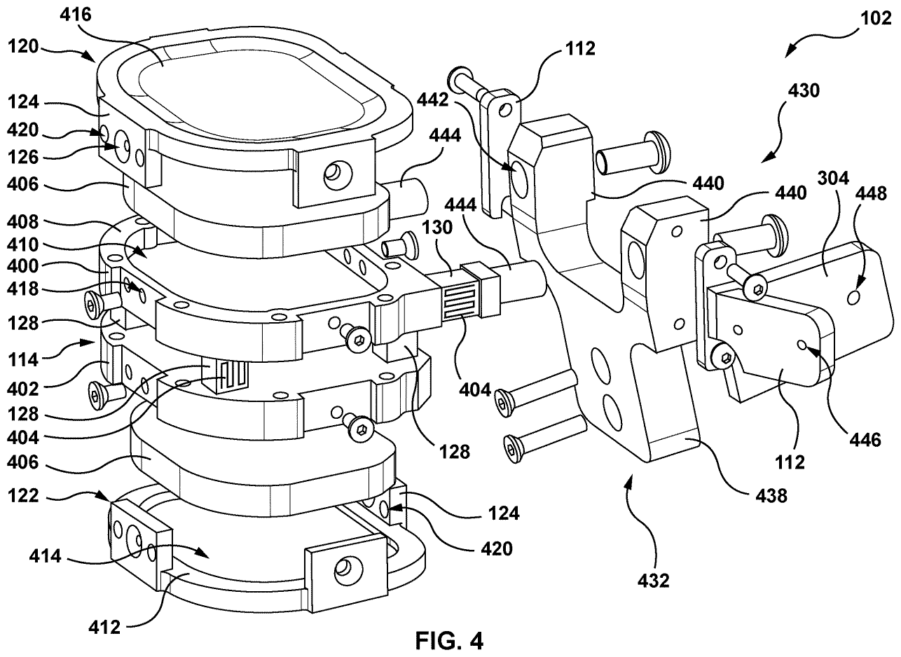

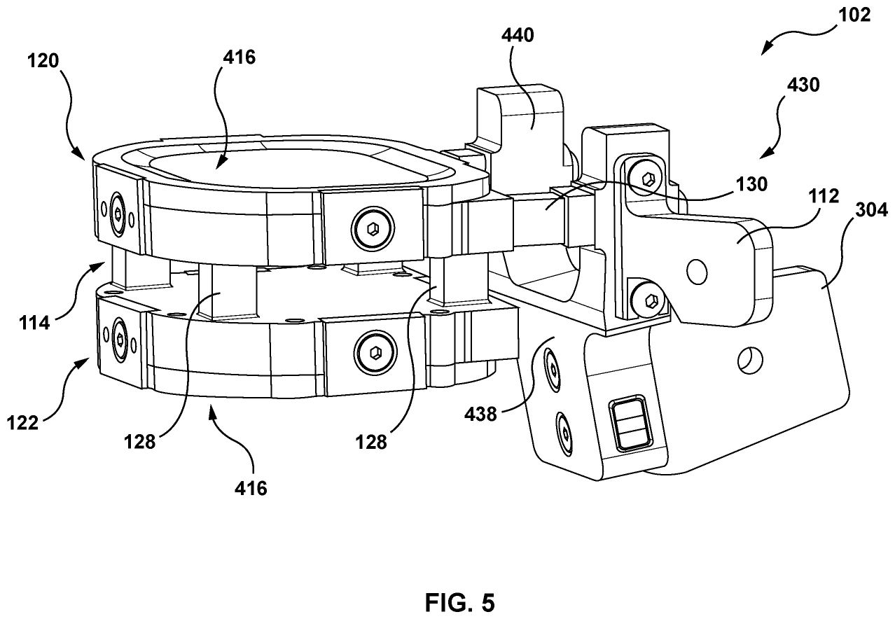

FIGS. 4 and 5 show an example artificial vertebra 102 that may be used to construct the biofidelic instrumented spine 100 discussed above. The above description of the spine 100 may be referenced for details not repeated here, with like reference numerals or terminology denoting like components.

The artificial vertebra 102 includes a main part 114, a first end plate 120, and a second end plate 122. The end plates 120, 122 are removably attachable to the main part 114, such as by screws. The first end plate 120 is attached to a first side (top) of the main part 114 and the second end plate 122 is attached to a second, opposite side (bottom) of the main part 114. The main part 114 and end plates 120, 122 may be made of rigid material, such as metal, and form a rigid body when connected together. For example, the main part 114 may be made of stainless steel and the end plates 120, 122 may be made of aluminum.

In this example, the main part 114 includes a pair of opposing generally planar bodies 400, 402 connected by posts 128. The posts 128 offset the planar bodies 400, 402 from each other. Four posts 128 may be used. In other examples, other numbers of posts 128, such as two, three, or five, may be used. A post 128 provides an instrumentation surface for attaching a sensor, such as a strain gauge 404. Any of the posts 128 may be provided with any number of strain gauges 404, each of which may be oriented in any useful orientation. The posts 128 concentrate forces experienced by the generally planar bodies 400, 402, so that sensor measurements may be more sensitive.

The main part 114 and end plates 120, 122 may be shaped to define voids to receive artificial cancellous bone 406. In this example, the upper planar body 400 of the main part 114 includes a ridge 408 that extends around its perimeter to define a recess 410. The upper end plate 120 includes a similar ridge to define a complementary recess (not shown) to cooperate with the recess 410 to define a void to receive artificial cancellous bone 406. Likewise, the lower planar body 402 of the main part 114 includes a ridge to define a recess (not shown) similar to the recess 410 of the upper planar body 400. The lower end plate 122 includes a similar ridge 412 to define a recess 414 to cooperate with the lower planar body 402 to define a void to receive artificial cancellous bone 406.

It should be noted that the upper and lower end plates 120, 122 have the same general shape and function. Features discussed for an upper end plate 120 may be provided to a lower end plate 122 and vice versa, unless otherwise mentioned.

The pieces of artificial cancellous bone 406 are shaped to be captured within the voids between the end plates 120, 122 and the main part 114. In this example, the artificial cancellous bone 406 is generally planar in shape with a rounded perimeter. The corresponding recesses in the end plates 120, 122 and the main part 114 are similarly shaped. The artificial cancellous bone 406 may be made of foam selected to simulate the properties of human cancellous bone.

The upper end plate 120 may include a disc-contact surface 416 shaped to contact an artificial intervertebral disc 104 (FIG. 1). The disc-contact surface 416 may be provided in a recess to securely hold the artificial intervertebral disc 104. Similarly, the lower end plate 122 may include a disc-contact surface (not shown) shaped to contact an artificial intervertebral disc 104.

The end plates 120, 122 may include tabs 124 with screw holes 126, each tab 124 being shaped to overlap a face of the main part 114 that has a complementary screw hole 418, so that screws may be used to attach the end plates 120, 122 to the main part 114.

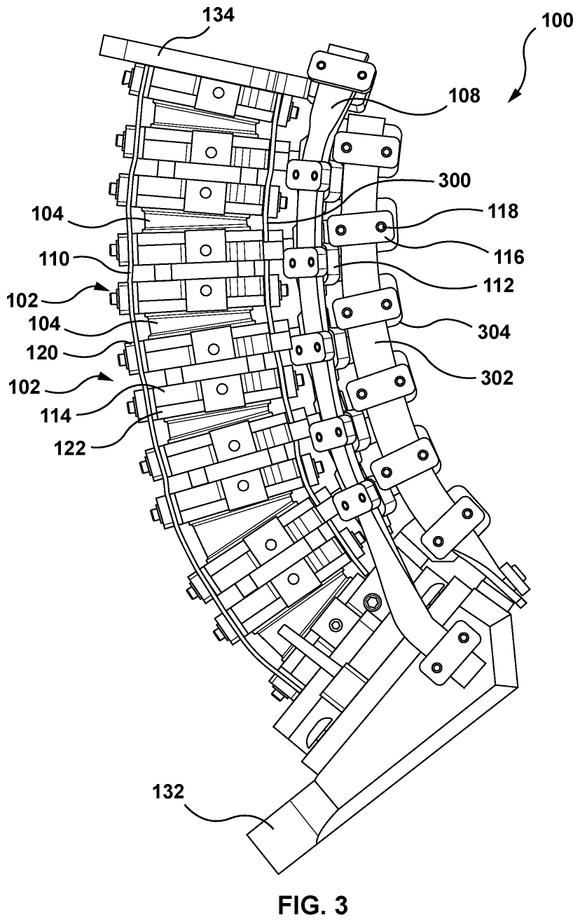

A tab 124 at the front of the artificial vertebra 102 may include an attachment point with screw holes 420, for example, to secure an artificial anterior longitudinal ligament 110 (FIG. 1). Likewise, a tab 124 at the rear of the artificial vertebra 102 may include an attachment point with screw holes 420, for example, to secure an artificial posterior longitudinal ligament 300 (FIG. 3).

The main part 114 of the artificial vertebra 102 may include a pair of artificial pedicles 130 extending in a rearward direction. An artificial pedicle 130 provides an instrumentation surface for attaching a sensor, such as a strain gauge 404. Any of the artificial pedicles 130 may be provided with any number of strain gauges 404, each of which may be oriented in any useful orientation.

The artificial vertebra 102 may further include a posterior component 430 to simulate additional posterior structures of human vertebrae and provide attachment points for additional artificial ligaments. The posterior component 430 may be a rigid assembly of parts, which may be fastened together by screws. The posterior component 430 may be made of metal, such as stainless steel or aluminum.

The posterior component 430 includes a bracket 432, a pair of artificial transverse processes 112, and an artificial spinous process 304.

The bracket 432 may be Y-shaped and may include a stem 438 to simulate a lamina and a pair of forks 440 each to simulate a superior articular process. Each fork 440 may include a hole 442 to receive a shaft 444 extending from an artificial pedicle 130 of the main part 114. Screws may be used to secure the shafts 444 within the holes 442 to attach the bracket 432 to the main part 114.

Each artificial transverse process 112 may have a planar shape that extends generally laterally and rearwardly from one of the forks 440 of the bracket 432 and includes an attachment point with screw holes 446, for example, for securing an artificial intertransverse ligament 108 (FIG. 1). The artificial transverse process 112 may be secured to the bracket 432 by screws. A clamp 116 and screws 118 (FIG. 1) may attach to the artificial transverse process 112 to sandwich the artificial intertransverse ligament 108 therebetween.

The artificial spinous process 304 may have a planar shape that extends generally rearwardly from the stem 438 of the bracket 432 and includes an attachment point with screw holes 448, for example, for securing an artificial interspinous and supraspinous ligament 302 (FIG. 3). The artificial spinous process 304 may be secured to the bracket 432 by screws. A clamp 116 and screws 118 (FIG. 3) may attach to the artificial spinous process 304 to sandwich the artificial interspinous and supraspinous ligament 302 therebetween.

The heights of different artificial vertebra 102 (e.g., L1, L2, etc.) may be varied to produce lumbar lordosis in the assembled spine 100. Variations of vertebrae height may be introduced by tapering the upper and lower end plates 120, 122 to achieve desired overall vertebrae dimensions. The relatively more complex main part 114 may be kept uniform to reduce complexity of the spine 100 and provide flexibility to modify vertebrae 102 geometries as required.

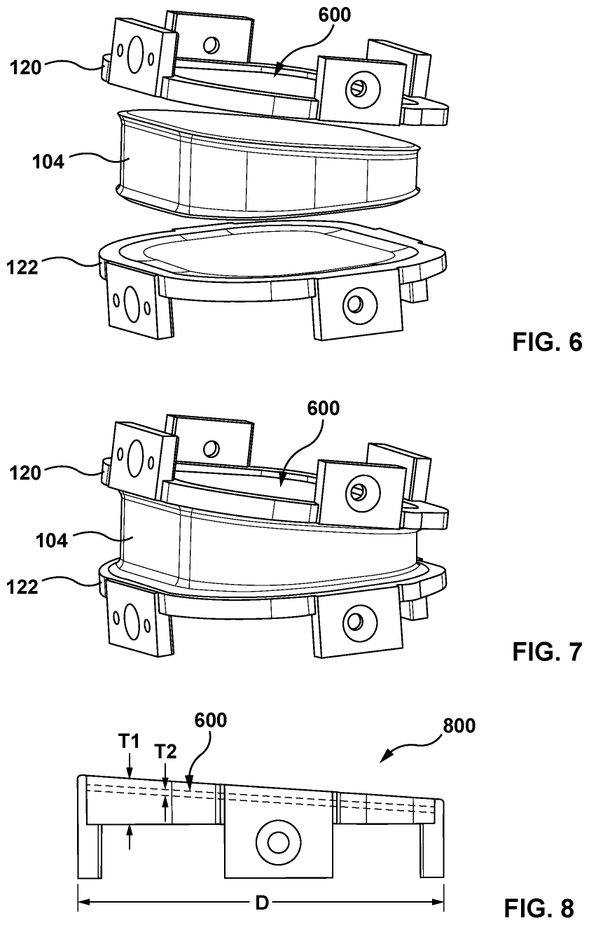

FIGS. 6 and 7 show example end plates 120, 122 and an example artificial intervertebral disc 104 that may be used to construct the biofidelic instrumented spine 100 discussed above. The above description of the spine 100 and artificial vertebra 102 may be referenced for details not repeated here, with like reference numerals or terminology denoting like components.

The ends plates 120, 122 may be made of metal, such as aluminum, and may have a diaphragm-like central planar region 600 having a thickness selected to provide deflection under loading and interaction with the artificial cancellous bone 406 (FIG. 4) for fidelity to human vertebrae. The central planar region 600 defines a disc-contact surface 416 (FIG. 4).

The artificial intervertebral disc 104 may be a generally wedge-shaped body made of molded homogeneous elastomer material with properties selected to provide the desired biofidelic range of motion and stiffness. The artificial intervertebral disc 104 may be bonded to the adjacent vertebrae end plates 120, 122 using an adhesive. The assembly of the artificial intervertebral disc 104 and end plates 120, 122 may be provided with a geometry that provides an intervertebral angle to produce lumbar spine lordosis and geometry.

FIG. 8 shows an example end plate 800 that may be used as any of the end plates 120, 122 discussed above.

An overall thickness T1 of the end plate 800 may change along an anterior-posterior depth D of the end plate 800 to provide a tapered shape that assists in simulating lordosis.

A thickness T2 of a central planar region 600 of the end plate 800 may be selected to provide deflection that interacts with adjacent artificial cancellous bone 406 (FIG. 4) to simulate the response of human cancellous bone.

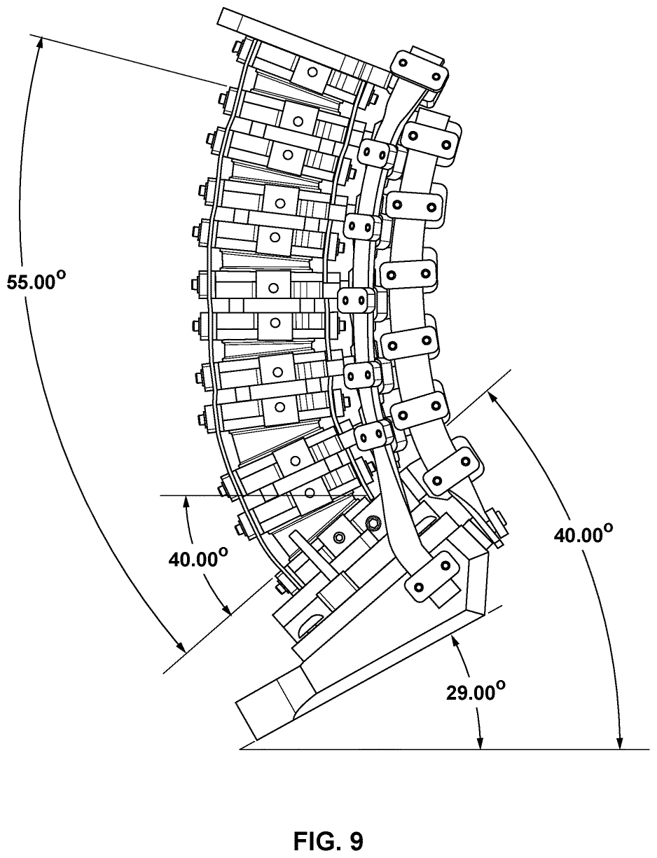

With reference to FIG. 9, the geometry of the artificial vertebrae main parts and end plates and the geometry of the artificial intervertebral discs may be selected so that the assembled artificial spine simulates, in a simplified manner, actual human lumbar spine morphology in the standing posture, as indicated by the example angles shown, which are based on published human morphology data. The biomechanical response of the artificial spine to different postures and loading conditions may be achieved by selecting properties for the artificial intervertebral discs and ligaments to be representative of the response of the human lumbar spine in these conditions. Geometry and response may be tailored, with reference to human morphology data, to suit specific implementations.

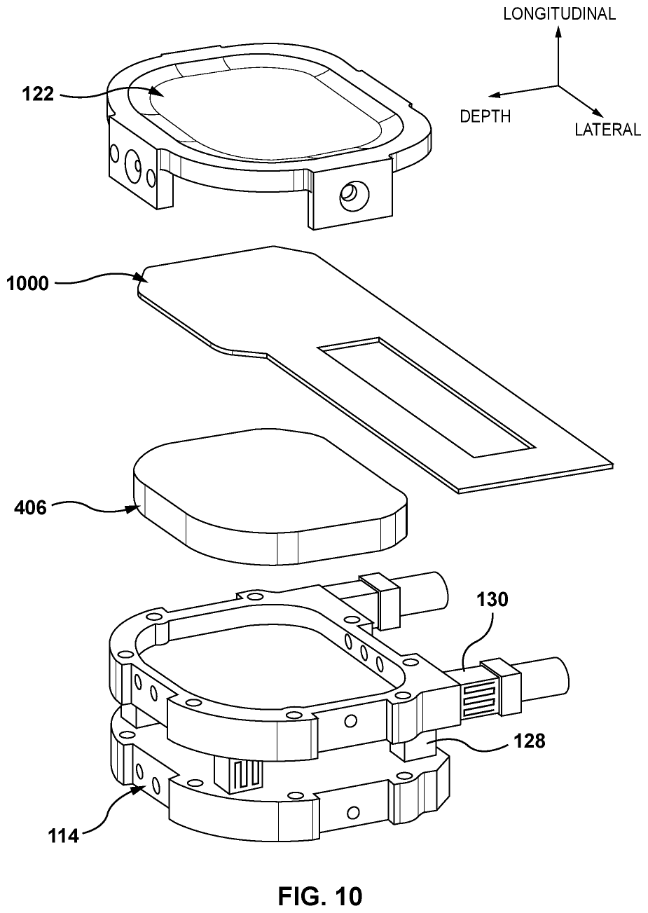

FIG. 10 shows the positioning of a pressure mapping sensor 1000, such as pressure mapping film (available from Tekscan™ of Norwood, MA, USA), in an artificial vertebra. The pressure mapping sensor may be sandwiched between an end plate 120 and artificial cancellous bone 406. The deflectable construction of the end plate 120 allows for the measurement of end plate pressure distribution. This allows measuring and mapping of the distribution of normal pressure applied by the end plate 120 and the artificial cancellous bone 406 as a reaction to the force on the artificial intervertebral disc 104 (FIG. 7) in contact with the end plate 120. A pressure mapping sensor 1000 may alternatively or additionally be provided between the other end plate 122 (FIG. 4) and the artificial cancellous bone 406.

A strain gauge may be adhered to any of the four exposed surfaces of the four posts 128 and the two artificial pedicles 130 of the main body 114. Strain gauges may be oriented to measure strain, and thus derive internal vertebral force, along any of the longitudinal, lateral, and depth (anterior-posterior) axes. Moment may be computed based on the known separation distance between posts 128 and between artificial pedicles 130.

For example, internal forces of the artificial vertebra along the longitudinal, lateral, and depth axes may be measured by strain gauges positioned on the posts 128. Moments about the lateral and depth axes may be computed from such forces based on the distances between posts 128. Pedicle loads from the posterior component 430 (FIG. 4) and ligaments attached thereto may be measured by strain gauges positioned on the artificial pedicles 130. Pedicle moments may be computed based on the measured loads and the distance between the artificial pedicles 130.

Sensors, such as pressure mapping sensors and strain gauges, provided to one or more vertebrae components provide for quantified understanding of how an external loading, such as an impact, is distributed along the lumbar spine and within the vertebral structure, which allows identifying loading modalities and injury mechanisms that may be associated with specific injury types, such as UBB injuries.

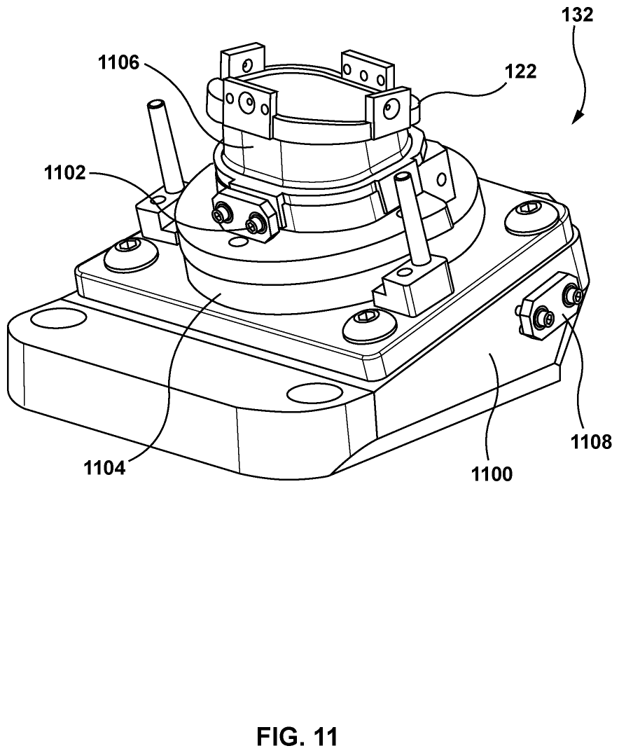

FIG. 11 shows a lower securing member 132 that may be used to construct the biofidelic instrumented spine 100 discussed above. The above description of the spine 100 and artificial vertebra 102 may be referenced for details not repeated here, with like reference numerals or terminology denoting like components.

The lower securing member 132 includes a pelvis adaptor 1100, an artificial sacral vertebra 1102 (i.e., S1), a load cell 1104, and an artificial intervertebral disc 1106 (i.e., L5-S1 disc).

The pelvis adaptor 1100 is shaped to provide an interface between the artificial spine 100 and the sacrum/pelvis of an anthropomorphic test device compatible with spine morphology in a seated posture. Other shapes of pelvis adaptor 1100 may be used for other postures.

The pelvis adaptor 1100 may include an attachment point 1108 for an artificial ligament. Any number of such attachment points 1108 may be provided.

The artificial sacral vertebra 1102 and artificial intervertebral disc 1106 are provided as an interface to the spine 100 with the pelvis adaptor 1100. The artificial intervertebral disc 1106 may be attached to a lower end plate 122 of an artificial vertebra 102 (i.e., L5).

The load cell may be used to measure loads at the base of the artificial spine 100. An example load cell is Denton™ model 1708.

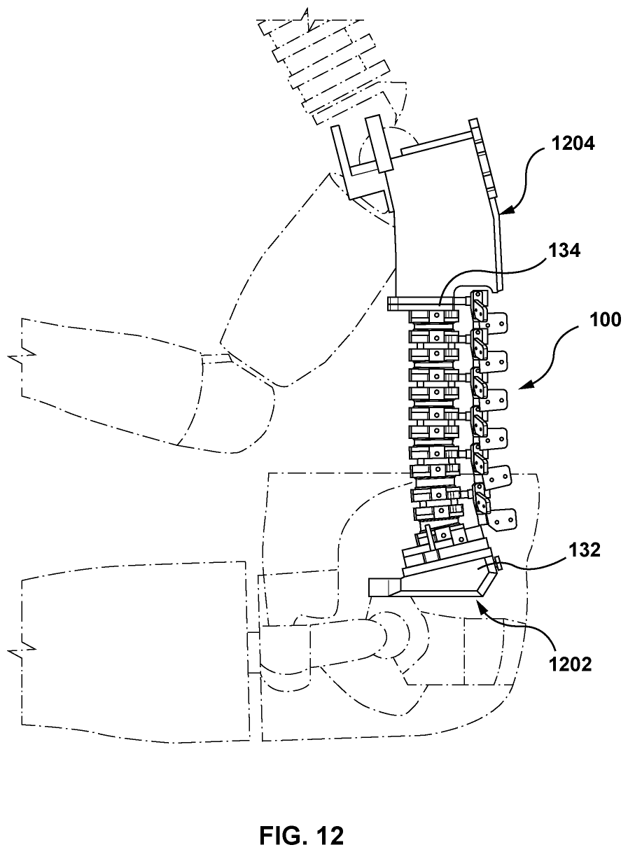

FIG. 12 shows a biofidelic instrumented spine 100 integrated into an anthropomorphic test device. The anthropomorphic test device may be a Hybrid III ATD. The above description of the spine 100 and lower securing member 132 may be referenced for details not repeated here, with like reference numerals or terminology denoting like components.

The artificial spine 100 may replace the known Hybrid III ATD lumbar spine component. A lower securing member 132 may be used to attach the artificial spine 100 to a pelvis component 1202 of the ATD. An upper securing member 134 may be used to attach the artificial spine 100 to a thoracic spine component 1204 of the ATD. The thoracic spine component 1204 may be a modified version of a Hybrid III thoracic spine component, in that the thoracic spine component 1204 is shortened to accommodate the artificial spine 100 because the artificial spine 100 is longer than the known Hybrid III ATD lumbar spine component.

Other modifications to the Hybrid III ATD or the artificial spine that may be useful to accommodate the artificial spine 100 with the Hybrid III ATD include removing vertebra T12 and IVD T12-L1, omitting the load cell from the lower securing member 132, shortening the pelvis component 1202 of the ATD, and reducing IVD height.

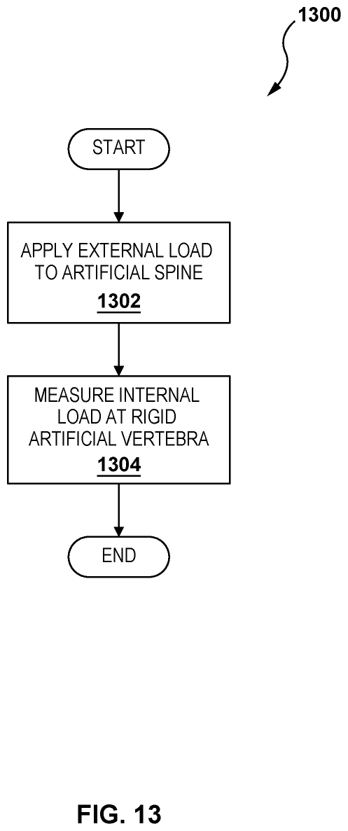

FIG. 13 shows an example method 1300 of measuring a vertebral load. The artificial spine 100 or another artificial spine using vertebrae 102 discussed herein may be used with the method 1300.

At block 1310, an external load is applied to the artificial spine. This may be done by installing the artificial spine 100 in an anthropomorphic test device (crash test dummy) and simulating a vehicle collision, UBB event, ejection seat deployment, or other event that may cause injury. In other examples, the artificial spine may be installed in a test bed and have a specific loading applied.

At block 1312, an internal load is measured using a sensor positioned at a rigid artificial vertebra of the artificial spine. As discussed above, any suitable number and type of sensors may be used such as strain gauges and pressure mapping film. The resulting measurements may be used to design vehicles, seats, vehicle armor, body armor, seating restraints, seatbelts, and other devices to reduce or minimize instances and degrees of spinal injury.

The techniques discussed above provide greater biofidelity of spine geometry and motion than known techniques. The artificial vertebrae and spine may be reused multiple times and provide repeatable behaviour at loads exceeding human tolerance levels. The components used to form the vertebrae and spine are relatively simple and provide for modularity and ease of replacement in case of damage. Moreover, the integration of instrumentation to the vertebrae allows for measurement of various internal loads to provide for improved prediction of injury mechanisms.

In the above, screws and adhesive are given as examples of suitable fasteners. It should be understood that any other types of suitable fasteners may be used. In addition, a component that described as being made of multiple parts may be made as one monolithic part, unless otherwise mentioned. Likewise, a monolithic component may be made with multiple attached or connected parts, unless otherwise mentioned. Terms such as “component,” “part,” “piece,” “element,” etc. are used interchangeably, unless otherwise indicated.

It should be recognized that features and aspects of the various examples provided above can be combined into further examples that also fall within the scope of the present disclosure. In addition, the figures are not to scale and may have size and shape exaggerated for illustrative purposes.

1. An artificial vertebra comprising:
a body of rigid material, the body including:
a disc-contact surface shaped to contact an artificial intervertebral disc; and
an instrumentation surface shaped to receive a sensor.

2. The artificial vertebra of claim 1, wherein the body further comprises:
a main part; and
an end plate that defines the disc-contact surface;
wherein the end plate is removably attachable to the main part.

3. The artificial vertebra of claim 2, wherein:
the end plate is a first end plate that is removably attachable to a first side of the main part; and
the body further comprises a second end plate that is removably attachable to a second side of the main part that is opposite the first side.

4. The artificial vertebra of claim 2, wherein:
the main part and the end plate are shaped to provide a void between the main part and the end plate when attached; and
the artificial vertebra further comprises artificial cancellous bone shaped to be captured within the void between the end plate and the main part.

5. The artificial vertebra of claim 2, wherein the end plate is tapered in a planar direction to provide lordosis to an assembly of artificial vertebrae that includes the artificial vertebra.

6. The artificial vertebra of claim 1, wherein:
the body further comprises a pair of opposing planar bodies connected by a post; and
the post includes the instrumentation surface.

7. The artificial vertebra of claim 1, wherein:
the body further comprises an artificial pedicle; and
the artificial pedicle includes the instrumentation surface.

8. The artificial vertebra of claim 1, wherein the body further comprises an attachment point for an artificial ligament.

9. The artificial vertebra of claim 1, further comprising a posterior component extending from the body and shaped to simulate additional posterior structure of a vertebra, the posterior component including an attachment point for an artificial ligament.

10. A biofidelic instrumented spine comprising:
a plurality of rigid artificial vertebrae arranged in a column;
a plurality of resilient artificial intervertebral discs, each of the plurality of resilient artificial intervertebral discs positioned between adjacent vertebrae of the plurality of rigid artificial vertebrae; and
a sensor positioned at a rigid artificial vertebra of the plurality of rigid artificial vertebrae to measure an internal loading of the rigid artificial vertebra.

11. The biofidelic instrumented spine of claim 10, further comprising an artificial ligament connected to each of the plurality of rigid artificial vertebrae.

12. The biofidelic instrumented spine of claim 10, wherein each of the plurality of rigid artificial vertebrae is made of metal.

13. The biofidelic instrumented spine of claim 10, wherein each resilient artificial intervertebral disc is made of elastomer.

14. The biofidelic instrumented spine of claim 10, wherein the sensor comprises a strain gauge.

15. The biofidelic instrumented spine of claim 10, further comprising artificial cancellous bone within the rigid artificial vertebra.

16. The biofidelic instrumented spine of claim 15, wherein the sensor comprises a pressure mapping sensor.

17. The biofidelic instrumented spine of claim 10, comprising a plurality of sensors each positioned at a respective rigid artificial vertebra of the plurality of rigid artificial vertebrae.

18. The biofidelic instrumented spine of claim 10, wherein each of the plurality of rigid artificial vertebrae comprises a tapered part to provide lordosis to the biofidelic instrumented spine.

19. A method comprising:
applying an external load to a biofidelic instrumented spine, the biofidelic instrumented spine comprising a plurality of rigid artificial vertebrae arranged in a column with resilient artificial intervertebral discs positioned therebetween, the plurality of rigid artificial vertebrae being connected by an artificial ligament; and
measuring an internal load using a sensor positioned at a rigid artificial vertebra of the plurality of rigid artificial vertebrae.

20. The method of claim 19, wherein the external load is configured to simulate an underbelly blast event.
